(19) United States
(12) Patent Application Publication
(10) Pub. No.: US 2010/0035268 A1
(43) Pub. Date: Feb. 11, 2010

(54) MATERIALS AND METHODS FOR SINGLE MOLECULE NUCLEIC ACID SEQUENCING

(76) Inventors: Joseph Beechem, Eugene, OR (US); Vi-En Choong, Carlsbad, CA (US); Theo Nikiforov, Carlsbad, CA (US)

Correspondence Address: LIFE TECHNOLOGIES CORPORATION;C/O INTELLEVATE, P.O. BOX 52050, MINNEAPOLIS, MN, 55402, US

(21) Appl. No.: 12/528,311

(22) PCT Filed: Feb. 21, 2008
(86) PCT No.: PCT/US08/54612

§ 371 Date: Aug. 21, 2009

(30) Foreign Application Priority Data
Feb. 21, 2007 (US) 60890976

(51) Int. Cl. C12Q 1/68

(52) U.S. Cl. 435/6

(57) ABSTRACT

Provided herein are methods and compositions for real time single molecule sequencing of short nucleotide sequences using nucleotide fluorescent semiconductor nanocrystals-conjugated nucleotide primers.

### CROSS-REFERENCES TO RELATED APPLICATIONS

This application claims benefit of priority to U.S. Provisional No. 60/890,976, filed Feb. 21, 2007.

### FIELD OF THE INVENTION

The present invention relates generally to real time single molecule sequencing. More particularly, the present invention relates to the use of semiconductor nanocrystals to provide detectable sequence information during synthesis of a polynucleotide.

### BACKGROUND OF THE INVENTION

DNA sequencing has traditionally been performed using large quantities of the target DNA molecule to be sequenced using resource and time intensive processes. Traditional Maxam-Gilbert sequencing involves the chemical cleavage of end-labeled fragments of DNA. The resulting fragments are then size separated by gel electrophoresis, and the sequence of the original end-labeled fragment are determined by analyzing the pattern of fragments produced by the gel. Read lengths using this approach are typically limited to approximately 500 nucleotides.

A more efficient sequencing method, Sanger-dideoxy sequencing, involves elongation of an end-labeled nucleotide primer with random incorporation of chain terminating dideoxy nucleotides in four separate DNA polymerase reactions. As with the chemically cleaved DNA fragments in the Maxam-Gilbert method, the extension products must be size separated by gel electrophoresis and the nucleotide sequence may be determined from analyzing the pattern of fragments in the gel. Originally performed with radionucleotide labeled primers, today the use of four different fluorescently labeled dideoxynucleotides enables the sequencing reactions to be size separated in a single gel lane, facilitating automated sequence determination. Read lengths utilizing this approach are limited to approximately 1000 nucleotides, and the process can take a few hours to half a day to perform.

U.S. Pat. No. 6,982,146 B1 (issued Jan. 3, 2006) describes DNA sequencing methods involving a polymerase carrying a donor fluorophore, and a mixture of nucleotides each carrying a distinguishable acceptor fluorophore. As the polymerase incorporates individual nucleic acid molecules into a complementary strand, a laser continuously irradiates the donor fluorophore. Emission from the polymerase is capable of stimulating any of the acceptor fluorophores.

Despite the improvements made to date, DNA sequencing still requires relatively large amounts of DNA substrate. All of the methods have significant limitations, such as the need for complex liquid handling step, short read-lengths, and overall complicated biochemistry. In addition, these approaches are not well-suited for rapid sequencing of nucleic acid molecules. Thus, there is a need in the art for rapid nucleic acid sequencing methods and compositions, for example real-time sequencing, from small amounts of target molecules, for example a single nucleic acid molecule.

### SUMMARY OF THE INVENTION

FRET-based methods are disclosed for sequencing single molecules of DNA. Donor-acceptor interactions between a semiconductor nanocrystal and a fluorophore enable the detection and identification of single bases as they are incorporated into a synthesized polynucleotide strand. More particularly, provided herein is a method for genotyping or sequencing a single target nucleic acid molecule, said method comprising: a) immobilizing onto a solid support a target nucleic acid sequence to form a solid support comprising more than one site or location each bearing only one single individual molecule of target nucleic acid sequence; b) contacting the solid support with a polymerase, a primer operably linked to at least one semiconductor nanocrystal, and at least one fluorescent terminally-labeled nucleotide polyphosphate; c) optically detecting a time sequence of incorporation of the fluorescently labeled nucleotide polyphosphates into the growing nucleotide strand at an active site complementary to the target nucleic acid, by detecting fluorescence resonance energy transfer (FRET) signals between the semiconductor nanocrystal and the fluorescent terminally-labeled nucleotide polyphosphate, wherein the identity of each fluorescent terminally-labeled nucleotide is determined by its fluorescent label, wherein the fluorescent label is then cleaved from the nucleotide upon incorporation into the growing strand; and d) genotyping or sequencing said single target nucleic acid by converting the sequence of the detected FRET signals detected during the polymerization reaction into a nucleic acid sequence.

In other embodiments, the disclosure provides a method for sequencing a nucleic acid molecule, comprising: (a) providing a reaction mixture comprising a primer annealed to a nucleic acid molecule, wherein a quantum dot is operably linked to the primer; (b) contacting the reaction mixture with a nucleotide polyphosphate and a nucleotide polymerase, wherein a label is operably linked to the nucleotide polyphosphate; (c) illuminating the reaction mixture; and (d) detecting the emission of light by FRET between the quantum dot and the label operably linked to the nucleotide polyphosphate. The disclosure also provides a method for sequencing a nucleic acid molecule, comprising: (a) providing a reaction mixture comprising a nucleic acid molecule; (b) contacting the reaction mixture with a primer complementary to the nucleic acid molecule, wherein a quantum dot is operably linked to the primer; (c) illuminating the reaction mixture; (d) contacting the reaction mixture with a nucleotide polyphosphate and a nucleotide polymerase, wherein the nucleotide polyphosphate comprises a label; (e) extending the primer with the nucleotide polyphosphate; and (f) detecting a signal from the label by FRET between the quantum dot and the label. Another embodiment includes a method for sequencing a nucleic acid comprising: (a) nonradiative transfer of energy from a donor fluorophore to an acceptor fluorophore, wherein the donor fluorophore is operably linked to a nucleotide primer and the acceptor fluorophore is attached to a nucleotide polyphosphate; (b) emission of light from the acceptor fluorophore; and (c) detection of the light emitted from the fluorophore.

The nucleic acid can be DNA, and the polymerase is a DNA or RNA polymerase. In other embodiments, the nucleic acid is RNA, and the polymerase is reverse transcriptase. The polymerase can be a Klenow fragment of DNA polymerase I, E. coli DNA polymerase I, T7 DNA polymerase, T4 DNA polymerase, Thermus aquaticus DNA polymerase, or Thermococcus litoralis, DNA polymerase. The primer can be extended by a plurality of nucleotides. In some embodiments, the primer is extended by less than 50 nucleotides. The primer can comprise at least 10 nucleotides, at least 20 nucleotides, at least 30 nucleotides, and sometimes at least 40 nucleotides.

In some embodiments, the semiconductor nanocrystal can act as a donor fluorophore and the fluorescent label on the nucleotide polyphosphate acts as the acceptor fluorophore. The fluorescent label or fluorophore can be selected from the group consisting of fluorescein, cyanine, rhodamine, coumarin, acridine, Texas Red dye, BODIPY, ALEXA, and a derivative or modification of any of the foregoing. In certain embodiments, the label operably linked or attached to the nucleotide, in certain embodiments, may be a quencher. Each nucleotide can be attached to a label that can be discriminated from the other base pairs by differences in spectral emissions, intensity and the like when participating in the FRET reaction.

Preferably, the fluorescent label or fluorophore is attached to the γ-phosphate of the nucleotide. In some embodiments, the fluorescent, terminally-labeled nucleotide polyphosphate has three or more phosphates. In other embodiments, the fluorescent terminally-labeled nucleotide polyphosphate has four or more phosphates. In some embodiments, the nucleotide polyphosphate is not terminally-labeled, but rather labeled on an internal phosphate, for example, the α-phosphate, the β-phosphate, or another internal phosphate.

The detection of incoming nucleotide polyphosphate and their base identification occurs in real-time or near real-time. In some embodiments, the method further comprises sequencing a second nucleic acid according to the method of claim 1 in parallel with sequencing the first nucleic acid.

In certain embodiments the quantum dot or nanocrystal is attached to the solid support. In other embodiments the primer or the nucleic acid to be sequenced may be attached to solid support. Any desired number of target molecules can be sequenced simultaneously while attached to the solid support. In some embodiments, the location of the individual molecules is addressable in the support. Any suitable solid support can be employed. In some embodiments, the solid support is glass, plastic, glass with surface modifications, silicon, metals, semiconductors, high refractive index dielectrics, nylon, nitrocellulose, PVDF, crystals, gels, and polymers. The solid support can be in any format including but not limited to a plate, microarray, sheet, filter, or beads.

The present disclosure also provides compositions useful for the sequencing of a nucleic acid molecule. One such composition is a reaction mixture comprising: (a) a primer, wherein a quantum dot is operably linked to the primer; (b) a nucleotide polymerase; and (c) a nucleotide polyphosphate, wherein a label is operably linked to the nucleotide polyphosphate. Another embodiment is a composition comprising: (a) a primer annealed to a target nucleic acid molecule, wherein a quantum dot is operably linked to the primer; (b) a nucleotide polymerase in contact with the nucleic acid molecule; and (c) a nucleotide polyphosphate in contact with the nucleotide polymerase, wherein a label is operably linked to the nucleotide polyphosphate.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from the claims and specification.

### DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1 shows a schematic of the nanocrystal-labeled primer sequencing reaction.

FIGS. 2A and B show a schematic of the synthesis of γ-labeled nucleotides.

FIG. 3 shows a representative image flow analysis.

FIG. 4 shows an exemplary time series and correlation analysis.

### DETAILED DESCRIPTION OF THE INVENTION

FRET-based methods provide significant advantages in sequencing polynucleotides. The sensitivity and accuracy of these methods permits single-molecule sequencing and reduces the number of handling steps in the process. Using semiconductor nanocrystals operably linked to an immobilized primer, sequence data can be generated in real time or near real time for small polynucleotides such as microRNA analysis, genotyping of discrete regions, SNP analysis, and the like.

The present disclosure provides compositions and methods for rapid sequencing of target nucleic acid molecules which utilize a nucleic acid primer labeled with a colloidal semiconductor nanocrystal, e.g, a quantum dot. The primer anneals to the target nucleic acid molecule, followed by polymerization from the 3′ end of the primer to incorporate one or more labeled nucleotides complementary to the target nucleic acid molecule. Fluorescence resonance energy transfer (FRET) between the quantum dot on the primer and a label on the incoming nucleotides that are incorporated into the complementary strand provides a detectable signal such that the identity of each incorporated nucleotide can be determined. This detection process can occur in real time or nearly in real time, and can be used to accurately and rapidly sequence at single nucleic acid molecule level.

The use of the word “a” or “an” when used in conjunction with the term “comprising” in the claims or specification may mean “one,” but it is also consistent with the meaning of “one or more,” “at least one,” and “one or more than one.”

As used in the claims and specification, the words “comprising” (and any form of comprising, such as “comprise” and “comprises), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

The practice of the present disclosure will employ, unless otherwise indicated, conventional techniques of molecular biology, microbiology and recombinant DNA techniques, which are within the skill of the art. Such techniques are explained fully in the literature. See, for example, Sambrook J and Russell D W, 2001, Molecular Cloning: A Laboratory Manual, Third Edition; Ausubel F M et al., eds., 2002, Short Protocols In Molecular Biology, Fifth Edition. All patents, patent applications, and publications mentioned herein, both supra and infra, are hereby incorporated by reference.

As used herein, the term “operably link” refers to chemical fusion or bond or an association of sufficient stability to withstand conditions encountered in the method of nucleotide sequencing utilized, between a combination of different molecules such as, but not limited to: between a linker and a functionalized nanocrystal; between a linker and a nucleotide; and the like. For example, a functionalized nanocrystal-labeled primer template, is performed in such a way that the resultant labeled primer can readily serve to initiate a polymerization reaction by a polymerase. Reactive functionalities comprise bifunctional reagents/linker molecules, free chemical groups (e.g., thiol, or carboxyl, hydroxyl, amino, amine, sulfo, etc.), reactive chemical groups (reactive with free chemical groups), and a combination thereof. Exemplary embodiments include but are not limited to those described in U.S. Pat. No. 6,326,144.

The term “linker” refers to a compound or moiety that acts as a molecular bridge to operably link two different molecules, wherein one portion of the linker binds to a functionalized nanocrystal, and wherein another portion of the linker binds to a nucleotide in the primer template. The two different molecules may be linked to the linker in a step-wise manner. There is no particular size or content limitations for the linker so long as it can fulfill its purpose as a molecular bridge suitable for use in strand synthesis. Linkers are known to those skilled in the art to include, but are not limited to, chemical chains, chemical compounds (e.g., reagents), and the like. The linkers may include, but are not limited to, homobifunctional linkers and heterobifunctional linkers. Heterobifunctional linkers, well known to those skilled in the art, contain one end having a first reactive functionality to specifically link a first molecule, and an opposite end having a second reactive functionality to specifically link to a second molecule.

The reactive functionalities of the linker are selected from the group consisting of amino-reactive groups and thiol-reactive groups. That is, the linker should be able to function to operably link by interacting with either a free thiol group or a free amino group present on either or both of the functionalized nanocrystal and the nucleotide to be linked. Depending on such factors as the molecules to be linked, and the conditions in which the method of strand synthesis is performed, the linker may vary in length and composition for optimizing such properties as stability, resistance to certain chemical and/or temperature parameters, and of sufficient stereo-selectivity or size to operably link the label to the nucleotide such that the resultant labeled nucleotide may serve as a template for the initiation of a polymerization reaction. Such linkers can be employed using standard chemical techniques. Such linkers are known to those skilled in the art to include, but are not limited to, amine linkers for attaching labels to nucleotide (see, e.g., U.S. Pat. No. 5,151,507); a linker preferably contain a primary or secondary amine for operably linking a label to a nucleotide; and a rigid hydrocarbon arm added to a nucleotide base (see, e.g., Science 282:1020-21, 1998).

Any suitable semiconductor nanocrystal can be used in the disclosed methods. See, e.g., U.S. Pat. Nos. 6,326,144; 5,990,479; 6,207,392; 6,306,610; and 6,221,602. In one embodiment, the semiconductor nanocrystal is a quantum dot (QD). In a specific embodiment, the QD is Q605. Quantum dots are a semiconductor nanocrystal with size-dependent optical and electronic properties. In particular, the band gap energy of a quantum dot varies with the diameter of the crystal. Useful quantum dots include those which are functionalized (a) to be water-soluble, and (b) to further comprise a nucleotide which is operably linked thereto. Desirable features of the basic quantum dots themselves include that the class of quantum dots can be excited with a single excitation light source resulting in a detectable fluorescence emission of high quantum yield (e.g., a single quantum dot having at a fluorescence intensity that may be a log or more greater than that a molecule of a conventional fluorescent dye) and with a discrete fluorescence peak. The quantum dots typically should have a substantially uniform size of less than 200 Angstroms, and preferably have a substantially uniform size in the range of sizes of from about 5 nm to about 20 nm.

In some embodiments, quantum dots have a core of CdX wherein X is Se or Te or S. CdX quantum dots can be passivated with an overlayering (“shell”) uniformly deposited thereon. Exemplary passivating shell can comprise YZ wherein Y is Cd or Zn, and Z is S, or Se. The quantum dots useful in the claimed methods are functionalized to be water-soluble nanocrystals. “Water-soluble” is used herein to mean that the nanocrystals are sufficiently soluble or suspendable in a aqueous-based solution including, but not limited to, water, water-based solutions, buffer solutions, that are used in one or more processes such as sequence determination, as known by those skilled in the art. In some embodiments, the CdX core/YZ shell quantum dots are overcoated with trialkylphosphine oxide, with the alkyl groups most commonly used being butyl and octyl.

Methods for synthesizing quantum dots suitable for fluorescence imaging in biological systems are well known. See, for example, Murray et al., 1993, J. Am. Chem. Soc. 115:8706-8715; Hines et al., 1996, J. Phys. Chem. 100:468-71; Peng et al., 1997, J. Am. Chem. Soc. 119:7019-29; U.S. Pat. No. 6,821,337. Alternatively, quantum dots are also available from commercial manufacturers such as QDOT® nanocrystals from Invitrogen (Carlsbad, Calif.).

For use in the disclosed methods, the semiconductor nanocrystal can have any suitable surface chemistry that permits the attachment of the nanocrystal or quantum dot to the biological molecule of interest, i.e., primer sequence. For example, quantum dots with a carboxyl-derivatized amphiphilic coating can be coupled to amines, hydrazines, or hydroxylamines using an EDAC-mediated reaction. Amino-derivatized coatings permit crosslinking with amine reactive groups such as isothiocyanates, succinimidyl esters and other active esters. Finally, quantum dots coated with covalently bound streptavidin or PEG enable linking to biotinylated molecules. See, e.g., U.S. Pat. Nos. 6,251,303; 6,274,323; and 6,306,610. Quantum dots with all of these surface chemistries, in a wide variety of emission wavelengths are available from Invitrogen (Carlsbad, Calif.) and other commercial manufacturers.

FRET, also known as Förster Resonance Energy Transfer, is a fluorescence imaging technique that allows investigators to tell when two fluorescently labeled molecules or moieties are in close proximity to each other. FRET occurs when a first, excited fluorophore, called a donor, non-radiatively transfers energy to a second fluorophore, called an acceptor, which may then emit a photon.

Importantly, FRET can only occur when the donor and acceptor are sufficiently close to each other, and FRET efficiency sharply decreases with distance (1/r⁶, where r=distance). The distance where FRET efficiency is 50% is termed R₀ (also known as the Förster distance) and is unique for each donor-acceptor combination. R₀ distances of 5 to 10 nm are typical for most donor-acceptor combinations. Given the steepness of the 1/r⁶ efficiency curve, for distances less than R₀, FRET efficiency is near maximal, and for distances greater than R₀, FRET efficiency is near zero. Consequently, in most biological applications, FRET effectively yields a binary, on-off type signal, indicating when the donor and acceptor are roughly within R₀ distance of each other.

Donor-acceptor pairs must be chosen such that there is overlap between the emission spectrum of the donor and excitation spectrum of the acceptor. Although the energy transfer from the donor to the acceptor does not involve emission of light, it may be thought of in the following terms: excitation of the donor produces energy in its emission spectrum which is then picked up by the acceptor in its excitation spectrum, leading to the emission of light from the acceptor in its emission spectrum. In effect, excitation of the donor sets off a chain reaction, leading to emission from the acceptor when the two are sufficiently close to each other.

In addition to spectral overlap between the donor and acceptor, other factors affecting FRET efficiency include the quantum yield of the donor and the extinction coefficient of the acceptor. The FRET signal may be maximized by selecting high yielding donors and high absorbing acceptors, with the greatest possible spectral overlap between the two.

The non-FRET signal of the donor must also be considered when designing a FRET detection system. Excited donor fluorophores not undergoing FRET will fluoresce, and care must be taken such that the non-FRET signal does not interfere with FRET signal corresponding to an incorporation event. Such cross-talk can occur in primarily two ways. First, donor fluorescence can excite the acceptor, leading to fluorescence from the acceptor, even when the donor and acceptor are not with R₀ of each other. Second, the donor fluorescence may leak into the detection channel for the acceptor fluorophore, swamping the FRET signal and making it difficult to detect. These problems are aggravated when the donor:acceptor ratio is skewed such that the number of donor fluorophores greatly exceeds the number of acceptors. FRET systems have a 1:1 donor:acceptor ratio may be preferred, but such a ratio may not be practicable in certain detection systems.

Additional information on FRET and parameters affecting FRET efficiency and signal detection may be found in Piston D W and Kremers G J, 2007, Trends Biochem. Sci. 32:407, which is incorporated herein in its entirety.

Quantum dots have been successfully used for FRET detection in biological systems. See, for example, Willard et al., 2001, Nano. Lett. 1:469; Patolsky F et al., 2003, J. Am. Chem. Soc. 125:13918; Medintz I L et al., 2003, Nat. Mater. 2:630; Zhang C Y, et al., 2005, Nat. Matter. 4:826. Quantum dots make particularly good FRET donors for several reasons. For example, quantum dot emission may be size-tuned to improve spectral overlap with any particular acceptor fluorophore, and quantum dots also have greater quantum yields and are less susceptible to photobleaching than traditional FRET donors. Together, these characteristics enable greater FRET efficiencies and make continuous monitoring (such as real-time monitoring) for FRET interactions possible.

Because quantum dots are larger than traditional organic fluorescent dyes, the size of the dot relative to the R₀ of the donor-acceptor pair should be taken into consideration. For dots size-tuned to emit in the visible light spectrum, the radius from the dot's energy transferring core to its surface typically ranges from 2 to 5 nm. Given typical R₀ distances of 5-10 nm, this means that acceptor fluorophores must be within a few nanometers of the quantum dot surface for efficient FRET between common donor-acceptor pairs. Larger quantum dots may have R₀ distances that will fall within the shell of the dot itself, precluding efficient FRET. These spatial constraints are especially important when the quantum dot is used to monitor interaction between a protein, nucleic acid, or some other molecule conjugated to the quantum dot surface and the acceptor molecule. Interaction between the conjugated molecule and the acceptor must position the acceptor fluorophore close enough to the quantum dot to allow FRET that is sufficient for detection.

The sequencing methods and compositions of the present disclosure utilize a nucleic acid primer, for example a single-stranded nucleotide primer, that is labeled with one or more quantum dots. Extension from the primer incorporates labeled nucleotides complementary to the target nucleic acid molecule. FRET between the semiconductor nanocrystal (the donor) attached to the primer and the label (the acceptor) on the nucleotides that are incorporated into the complementary sequence results in a detectable signal identifying each incorporated nucleotide. Upon incorporation, the label can be released from the nucleotide, thereby eliminating the FRET signal between the donor and acceptor. In other embodiments, the acceptor signal from the incorporated nucleotide is quenched.

The sequence of any suitable nucleic acid molecule can be determined using the disclosed methods. Such sequences include, but are not limited to single-stranded DNA, double-stranded DNA, single stranded DNA hairpins, DNA/RNA hybrids, RNA with an appropriate polymerase recognition site, and RNA hairpins.

The nucleic primer can be of any suitable length. The length is typically determined by the specificity desired for binding the complementary template as well as the stringency of the annealing and reannealing conditions employed. The primer can comprise ribonucleotides, deoxynucleotides, nucleotide analogs or combinations thereof. For example, the nucleic acid primer may comprise ribonucleotide, deoxyribonucleotide, modified ribonucleotide, modified deoxyribonucleotide, peptide nucleic acid, modified peptide nucleic acid, modified phosphate-sugar backbone oligonucleotide, and other nucleotide and oligonucleotide analogs. As used herein, the term nucleotide encompasses those listed above as well as other nucleotide analogs. The nanocrystal-labeled primer can be either synthetic or produced naturally by primases, RNA polymerases, or other oligonucleotide synthesizing enzymes. The primer may be any suitable length including at least 5 nucleotides, 5 to 10, 15, 20, 25, 50, 75, 100 nucleotides or longer in length. The semiconductor nanocrystal or quantum dot may be conjugated or attached to the nucleotide primer through a variety of chemistries that form suitable linkages such that they accommodate the nucleic acid sequencing reaction. See, e.g., U.S. Pat. No. 6,221,602. Typically, the semiconductor nanocrystal is bound to the 3′ end of the primer, although the nanocrystal may be operably linked at any location on the primer. One or more semiconductor nanocrystals may be operably linked to the primer.

In some embodiments, the nucleic acid primer, and/or the target nucleic acid molecule may be attached to a substrate. The target nucleic acid can be attached to a support by immobilization of the semiconductor nanocrystal-labeled primer or the single-stranded or double-stranded target nucleic acid molecule. If a single stranded target nucleic acid molecule is employed, it is then hybridized to the solid-support attached, nanocrystal-labeled primer. When a double stranded molecule is employed, the semiconductor nanocrystal label is attached to the 3′ end of the primer sequence. Either the nanocrystal-labeled primer is hybridized to the immobilized target nucleic acid molecule, to form a primed target nucleic acid molecule complex suitable for initiation of a polymerization reaction or a recognition site for the polymerase is created on the double stranded template (e.g., through interaction with accessory proteins, such as a primase). The polymerase extends the nanocrystal-labeled primer by adding fluorescent labeled nucleotides to the primer at the active site that are complementary to the nucleotide of the target nucleic acid at the active site. The nucleotide analog added to the oligonucleotide primer as a result of the extending step is identified by optical detection and characterization of the FRET signal. Typically, the primer is extended at least 5 nucleotides, at least 10 nucleotides, at least 20 nucleotides, at least 30 nucleotides, at least 40 nucleotides, at least 50 nucleotides, at least 55 nucleotides, at least 60 nucleotides, at least 70 nucleotides, at least 80 nucleotides, at least 90 nucleotides or at least 100 nucleotides. The primer can be in any suitable form such as a single stranded molecule and a hairpin.

Any suitable materials may be used for the solid support. Exemplary materials include, but are not limited to, glass, plastic, glass with surface modifications, silicon, metals, semiconductors, high refractive index dielectrics, nylon, nitrocellulose, PVDF, crystals, gels, and polymers. The solid support can be in any format including plate, microarray, sheet, filter, and beads. Techniques for binding the target nucleic acid molecule and/or primer to the substrate are determined by the materials employed. For example, binding partners such as streptavidin can be employed with biotinylated template or primer. Reversible or irreversible binding between the support and either the nanocrystal-labeled primer or the target nucleic acid sequence can be achieved with the components of any suitable covalent or non-covalent binding pair. Other such suitable immobilization approaches for immobilizing can include an antibody(or antibody fragment)-antigen binding pair and photoactivated coupling molecules. Generally, suitable immobilization can be applied to the support by conventional chemical and photolithographic techniques which are well known in the art and include standard chemical surface modifications of the solid support, and support incubation using differential temperatures and media.

Any suitable nucleotide polymerase known in the art may be used including thermostable polymerase or a thermally degradable polymerase. Exemplary polymerases include, but are not limited to polymerases isolated from Thermus aquaticus, Thermus thermophilus, Pyrococcus woesei, Pyrococcus furiosus, Thermococcus litoralis, Thermotoga maritima, E. coli DNA polymerase, the Klenow fragment of E. coli DNA polymerase, T4 DNA polymerase, T7 DNA polymerase, E. coli T7, T3, SP6 RNA polymerases and AMV, M-MLV and HIV reverse transcriptases. In one example, reaction conditions for the Klenow fragment of DNA polymerase I typically include a buffer comprising 10 mM MgCl₂ and 50 mM NaCl at pH 8.0, incubated at room temperature to 37° C. Reaction conditions for other nucleotide polymerases are well known in the art and available in suitable molecular biology protocol texts, such as Sambrook J and Russell D W, 2001, Molecular Cloning: A Laboratory Manual, Third Edition or Ausubel F M et al., eds., 2002, Short Protocols In Molecular Biology, Fifth Edition, which are incorporated herein by reference.

The fluorescent label or fluorophore can be attached to the incoming nucleotide using any suitable linking chemistry. Such attachment may include a bridging linker to the nucleotide. Typically, the fluorescent label is attached to the terminal phosphate of the nucleotide. The nucleotide can have three or more phosphates. See, e.g., U.S. Pat. No. 7,041,812. In some embodiments, a single fluorescent label is linked to the terminal phosphate. As the incoming fluorescently-labeled nucleotide is incorporated, the phosphates are cleaved between the α and β phosphates, releasing the γ-linked labeled phosphate. Any suitable fluorophore that can participate as an acceptor for the semiconductor nanocrystal may be employed. In certain embodiments, the label is a fluorescent label. The fluorescent label may be a fluorescein, cyanine, rhodamine, coumarin, acridine, Texas Red dye, BODIPY, Alexa Fluor, or a derivative or modification of any of the foregoing. Alexa Fluor dyes available from Molecular Probes (Eugene, Oreg.) are available in emission wavelengths spanning the visible and infrared spectrum. In other embodiments, the label may be a quencher. Quenchers are useful as acceptors in FRET applications, because they produce a signal through the reduction or absence of fluorescence from the donor fluorophore. As with conventional fluorescent labels, quenchers have an absorption spectrum and large extinction coefficients, however the quantum yield for quenchers is extremely reduced, such that the quencher emits little to no light upon excitation. For example, in a FRET detection system, illumination of the donor fluorophore excites the donor, and if an appropriate acceptor is not close enough to the donor, the donor emits light. This light signal is reduced or abolished when FRET occurs between the donor and the appropriate quencher acceptor, resulting in little or no light emission from the quencher. Thus, interaction or proximity between a donor and quencher-acceptor may be detected by the reduction or absence of donor light emission. For an example of the use of a quencher as an acceptor with a quantum dot donor in a FRET system, see Medintz, I L et al. (2003) Nat. Mater. 2:630. Examples of quenchers are the QSY dyes available from Molecular Probes (Eugene, Oreg.).

The sequencing reaction is initiated by the addition of a suitable polymerase and labeled nucleotides. Suitable temperatures and the addition of other components such as divalent metal ions can be determined based on the target nucleic acid sequences. Illumination of the reaction site permits observation of the FRET reactions that mark the nucleotide incorporation. Polymerization of the primer by the nucleotide polymerase incorporates nucleotides from the reaction mixture into the elongating primer strand. Polymerase-catalyzed-extension adds incoming nucleotides onto the free 3′-OH end of the primer, such that the strand grows in the overall 5′ to 3′ direction. The addition of the incoming nucleotide results in formation of a phosphodiester bond between the 3′-OH and the α-phosphate of the incoming nucleotide, cleaving and releasing remaining Pᵢs from the nucleotide.

The identity of the incoming nucleotide is generally specified through Watson-Crick base-pairing with the next unpaired nucleotide on the template strand, such that a nucleotide comprising the nitrogenous base adenine (A) will base pair with a nucleotide comprising the nitrogenous base thymine (T), and a nucleotide comprising the nitrogenous base guanine (G) will base pair with a nucleotide comprising the nitrogenous base cytosine (C). When the elongating nucleotide strand is a ribonucleic acid (RNA) strand, a nucleotide comprising the nitrogenous base uracil (U) is substituted for T. Repeated rounds of nucleotide polymerase mediated extension results in the synthesis of a nucleic acid strand complementary in sequence to the template strand. Thus, applying Watson-Crick base-pairing rules, the sequence of the template strand—the nucleic acid to be sequenced—may be determined from the identities of the nucleotides incorporated into the primer strand.

The identities of the incorporated nucleotides may be determined rapidly, for example in real-time or near real-time, as extension of the primer strand occurs, through FRET interactions between the semiconductor nanocrystal or quantum dot (i.e., the donor) attached to the primer and a label (i.e., an acceptor) attached to the incoming nucleotides as they are incorporated into the complementary strand. The nucleotides used for extension of the primer in the present disclosure are labeled with either a fluorescent label, a quencher, or some combination thereof. In some embodiments, the label is attached to a phosphate, for example the β-phosphate, the γ-phosphate, or the terminal phosphate of the nucleotide, such that the label is separated from the nucleotide upon incorporation into primer strand by the nucleic acid polymerase. In other embodiments, the label is attached to the α-phosphate, the nitrogenous base, or the sugar of the nucleotide and used in combination with a quencher.

As discussed below, a number of labeling and detection strategies are available to determine the identity of the nitrogenous base of the incoming nucleotides. All of these strategies rely on FRET between the semiconductor donor attached to the primer and the fluorescent label and/or quencher acceptor attached to the incoming nucleotide. In the present disclosure, the quantum dot donor is excited by illumination with light of an appropriate excitation wavelength, as required by the excitation spectrum of the quantum dot. Given the exceptional photostability of quantum dots, continuous excitation without photobleaching is possible. As the nucleotide polymerase incorporates incoming nucleotides complementary to the target nucleic acid molecule into the primer strand, the label attached to the nucleotide is brought into close proximity with the quantum dot. When the distance between the quantum dot and label decreases to approximately 1.0 to 1.5×R₀ or less, FRET efficiency increases sufficiently to trigger detectable FRET between the quantum dot and label, either through the emission of light from the label or quenching of the quantum dot's light emission.

Detection of the FRET signal and spectral resolution permitting discrimination between the various nucleotide signals can be achieved using any suitable method including spectral wavelength analysis, correlation/anti-correlation analysis, fluorescent lifetime measurement, and fluorophore identification. Suitable techniques for detecting the emissions include confocal laser scanning microscopy, Total Internal Reflection Fluorescence (TIRF) and other forms of fluorescence microscopy.

In certain embodiments, the label is attached to a phosphate that is cleaved by the polymerase from the nucleotide upon incorporation into the complementary sequence, for example the β-phosphate, the γ-phosphate, or the terminal phosphate of the incoming nucleotide. By cleaving the phosphate and releasing the label upon incorporation of the incoming nucleotide, the FRET signal between the quantum dot and the label ceases after the nucleotide is incorporated and the label diffuses away. Thus, in these embodiments, a FRET signal is generated as each incoming nucleotide hybridizes to a complementary nucleic acid in the target nucleic acid molecule, and upon incorporation of the nucleotide into the elongating primer strand, the label is released and the FRET signal ends. By releasing the label upon incorporation, successive extensions can each be detected without interference from nucleotides previously incorporated into the complementary strand.

As the nucleotide polymerase continues to incorporate nucleotides into the elongating primer strand, the distance between the quantum dot and the site of nucleotide incorporation increases. Once the site of nucleotide incorporation is more than approximately 1.0 to 1.5×R₀ away from the quantum dot donor, FRET between the quantum dot and label becomes unlikely, and additional nucleotide incorporation events will be beyond the detection capabilities of the system. The Förster distance (R₀) depends in part on the specific combination of FRET donor and acceptor used. Thus, the number of nucleotides that may be sequenced with this approach is dependant on the combination of donor and acceptor used; the greater the Förster distance for a particular donor-acceptor pair, the greater the read length of this sequencing approach. In some embodiments up to about 10, 20, 30, 40, 50, 75 or 100 nucleotides may be sequenced using the methods and compositions disclosed herein.

A number of labeling and detection strategies are available for base discrimination using the FRET technique. For example, different fluorescent labels may be used for each nucleotide in the reaction mixture (for each type of nucleotide present in the extension reaction), with discrimination between the different labels based on the wavelength and/or the intensity of the light emitted from the fluorescent label.

A second strategy involves the use of fluorescent labels and quenchers. In this strategy, certain nucleotides in the reaction mixture are labeled with a fluorescent label, while the remaining nucleotides are labeled with one or more quenchers. Alternatively, each of the nucleotides in the reaction mixture is labeled with one or more quenchers. Discrimination of the nucleotide bases is based on the wavelength and/or intensity of light emitted from the FRET acceptor, as well as the intensity of light emitted from the FRET donor. If no signal is detected from the FRET acceptor, a corresponding reduction in light emission from the FRET donor indicates incorporation of a nucleotide labeled with a quencher. The degree of intensity reduction may be used to distinguish between different quenchers.

A third strategy involves modulating FRET efficiency by varying the distance between the quantum dot donor and the fluorescent label or quencher acceptor. In this strategy, the same type of fluorescent label or quencher may be used, however, the distance between the quantum dot and the label is varied for each nucleotide to be identified, causing a modulation of FRET efficiency. The distance may be varied through the structure of the nucleotide itself, the position of the fluorescent label or quencher on the nucleotide, or the use of spacers or linkers during attachment of the fluorescent label or quencher to the nucleotide. Modulation of FRET efficiency results in a detectable modulation of emission intensity or quenching.

In another strategy, FRET efficiency may be modulated by varying the number of fluorescent labels or quenchers attached to each incoming nucleotide. In this strategy, differing numbers of the same fluorescent label or quencher are attached to each nucleotide. For example, one fluorescent label may be attached to A, two to T, three to G, and four to C. Increasing the number of acceptors relative to the quantum dot donors increases FRET efficiency and quantum yield, such that base discrimination may be based on the intensity of light emission from the acceptor(s) or the reduction of light emission from the quantum dot donor(s).

Any combination of the above described labeling and detection strategies may be employed together in the same sequencing reaction. Depending on the number of distinguishable fluorescent labels and quenchers used in any of the above strategies, the identities of one, two, or four nucleotides may be determined in a single sequencing reaction. Multiple sequencing reactions may then be run, rotating the identities of the nucleotides determined in each reaction, to determine the identities of the remaining nucleotides. In some embodiments, these reactions may be run at the same time, in parallel, to allow for complete sequencing in a reduced amount of time.

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### Examples

### Example 1

### Preparation of γ-Phosphate Labeled TTP

An Alexa Fluor 647 dye labeled TTP was synthesized by using a carbodiimide condensation reaction between Alexa Fluor 647 hydrazide (Invitrogen Corp.; Carlsbad, Calif.) and unlabeled TTP. The reaction was conducted in the presence EDC (1-ethyl-3-(3-dimethylaminopropyl)carbodiimide hydrochloride) and MES (2-(N-morpholino)ethanesulfonic acid) at a pH of 5.5. At this pH, the majority of the hydrazide groups are unprotonated, and thus highly reactive towards the activated carbodiimide. The product was purified using HPLC using a C18 reverse phase column, with detection at 260 nm and 598 nm.

40 microliters of a 100 mM solution of TTP was mixed with 60 microliters of 8.3 mM Alexa Fluor 647 hydrazide. 85 microliters of 500 mM MES, pH 5.5 was added to the mixture. Finally, 5.6 mg of solid EDC was added to the mixture. Aliquots were analyzed by HPLC (reverse phase C18 column, using a gradient of acetonitrile in triethylammonium acetate pH 7.0). The appearance of the product peak eluting at about 8.9 minutes was monitored over time, and when two sequential injections did not reveal any additional product accumulation (approximately 85 minutes), the whole mixture was subjected to HPLC purification, using the same column and a modified elution gradient. The peak corresponding to the product was collected, the solution concentrated in a Speed Vac, and the product desalted on a C18 reverse phase cartridge, pre-equilibrated with 70% acetonitrile, washed with water. The colored reaction product was retained on the column, the column was washed extensively with water, and the product was eluted with 70% acetonitrile in water. The acetonitrile was removed in vacuo, the concentration of the product determined by its absorbance at 650 nm and its purity evaluate by running an analytical HPLC. The product was stored at −20° C.

A gamma-labeled dCTP was prepared in an analogous manner as described above using Alexa Fluor 647 hydrazide.

### Example 2

### FRET Detection of Polymerase Extension Reactions Using Nanocrystal-Labeled Hairpins

The self-complementary, hair-pin loop oligonucleotide (TTTTTGAGGGTGACAGGTTTTTCCTGTCACCX; where X is amino modifier C6 dC; SEQ ID NO:1) was covalently attached to the surface of PEG amine modified Qdot nanocrystals (Invitrogen Corp.; Carlsbad, Calif.) using SMCC based conjugation. The 3′ end of SEQ ID NO:1 dictates the enzymatic incorporation of a TTP followed by a dCTP. Incorporation of the dCTP is possible only after insertion of TTP.

The resulting nanocrystal conjugate was mixed with solutions of different Alexa Fluor dye-labeled dNTPs, such as labeled dCTP or labeled TTP. The dyes were attached to the heterocyclic base of the triphosphates, and become a permanent part of the enzymatic extension product on the nanocrystal.

Polymerization was initiated by addition of Klenow polymerase, and fluorescence emission of the solution was recorded in real-time using a plate reader (Molecular Devices Corp.; Sunnyvale, Calif.) set at an excitation wavelength of 450 nm. Emission was detected at two wavelengths, corresponding to the Qdot nanocrystal and Alexa Fluor fluorophore emissions. All reactions were performed in triplicate.

Addition of only labeled dCTP did not result in any increase in emitted fluorescence, indicating no extension (as expected). Addition of Alexa Fluor 647 labeled TTP only resulted in an increase in RFU from about 25 to 45-50 within 100 seconds. The RFU remained steady after that time.

Addition of labeled TTP to samples initially containing only labeled dCTP showed primer extension by an increase in RFU. This same result was obtained by addition of unlabeled “cold” TTP to samples initially containing only labeled dCTP. In this case, the FRET observed will be based upon the incorporation of the labeled dCTP.

### Example 3

### FRET Detection Using Nanocrystal-Labeled Primer

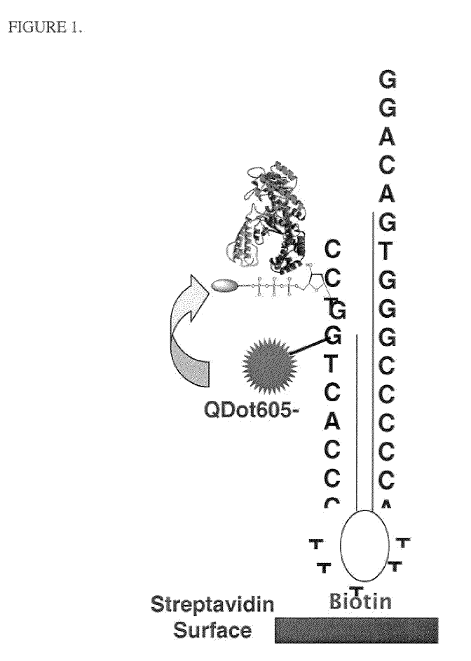

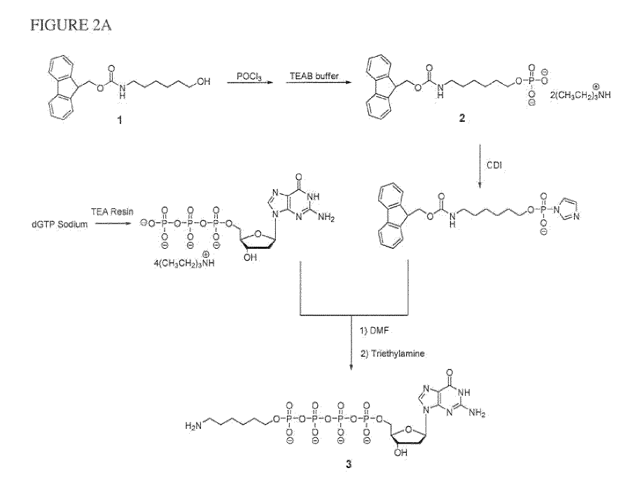

Conjugation of Q605 nanocrystal with primer oligonucleotide. This quantum dot-oligo conjugate was prepared in a two step procedure. First, the nanocrystals were reacted with adipic hydrazide and EDC, a water-soluble carbodiimide. In the second step, these modified nanocrystals were reacted with an aldehyde-modified hairpin-type oligonucleotide. See, e.g., FIG. 1.

Step 1: 170 μL of 300 mM adipic hydrazide and 240 μL of 500 mM MES pH 5.5 buffer were added to 100 μL of 8 μM Qdot 605 PEG 2000 amine dots. Then, 2.8 mg of solid EDC was added, the mixture mixed and allowed to stand at room temperature for 2 hours. The product was isolated by several rounds of ultrafiltration through a suitable ultrafiltration device using 250 mM MES pH 5.5 buffer.

Step 2: The adipic hydrazide derivatized quantum dots from Step 1 were mixed with a 10-15 fold molar excess of aldehyde-modified hairpin oligo (162). The mixture was kept at room temperature for 12 hours, followed by concentration to approx. 25 μL by ultrafiltration. The desired Qdot-oligo conjugate was purified from the excess free, unconjugated oligo by size-exclusion chromatography on Superdex 200 (GE Healthcare), equilibrated with phosphate buffered saline buffer pH 7.4.

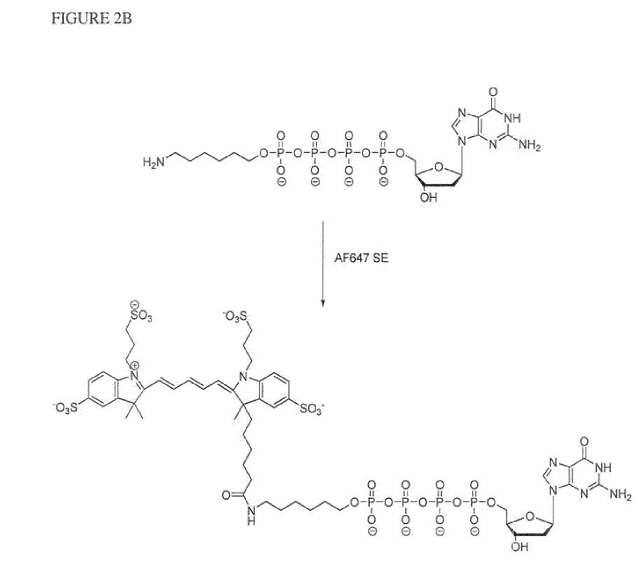

Synthesis of AF647-γ-deoxyguanosine-tetraphosphate. The synthetic route of the single-AF647 labeled dG tetraphosphate is illustrated in schemes 1 (FIG. 2A) and 2 (FIG. 2B).

Synthesis of compound 2: Compound 1 (678 mg, 2 mmol) was suspended in trimethyl phosphate (5 mL) and cooled to 0° C. POCl₃ (280 μL) was added to the stirred mixture under argon. The mixture was warmed up and stirred at room temperature overnight. The reaction was quenched by adding slowly 4 mL of TEAB buffer (1 M) at 0° C. Triethylamine was added to adjust the pH to pH 7.0. The solvent was evaporated and the residue was purified by column chromatography on silica gel, eluting with 10% H₂O/CH₃CN. After evaporation of the solvent, the solid was dissolved in water. The pH of the solution was adjusted to pH 7 with TEAB buffer (1 M), followed by coevaporation with methanol. Yield: 400 mg of compound 2.

Synthesis of compound 3. The sodium salt of dGTP (20 mg) was converted into its triethylammonim salt by passing a triethylammonium resin and dried in high vacuum. Compound 2 (42 mg) was dissolved in 2 mL of dry DMF. Carbonyldiimidazole (CDI) (65 mg) was added and the solution was stirred for 4 hours at room temperature, followed by the addition of anhydrous methanol (18 μL) and stirred for a further hour. The dried dGTP triethylammonium salt was dissolved in dry DMF (2 mL), and to this solution was added the prepared phosphoimidazolate solution of 2 under argon. The mixture was stirred under argon overnight. Triethylamine (1 mL) was added and stirred for 4 hours. The solvent was evaporated, washed with CHCl₃, dissolved in water and purified by sephadex A-25 DEAE ion exchange chromatography, eluting with a linear gradient of 0.05 M to 0.6 M TEAB buffer. After coevaporation with methanol and lyophilization, ca. 5 mg of compound 3 was obtained.

Synthesis AF647-dGP4. To a solution of AF647 SE (2 mg), compound 3 (1 mg) and Et₃N (5 μL) in DMF (300 μL) was added 150 μL of H₂O. The solution was stirred at room temperature until the completion of the reaction (ca. 1 hour). The product was purified by column chromatography on sephadex LH-20, eluting with water. The desired fraction was concentrated to ca. 500 μL and stored at −20° C.

Live single-molecule template-directed polymerization assay. Extension reactions were performed in 8-lane flow cells fabricated from 3 mm thick acrylic plastic, 3M tape, and 20×60×0.17 mm surface modified cover glass (PEG/PEG-biotin modification, MicroSurfaces, Inc., Minneapolis, Minn.). Flow cell lanes were first coated with streptavidin (200 μg/mL in 1% (w/v) BSA in phosphate buffered saline (4 mM phosphate pH 7.2, 150 mM NaCl) for 30-60 min, then washed by pipetting 200 μL PBST (PBS+0.1% (v/v) Tween-20) through the lanes and repeating for a total of five washes per lane. Biotinylated-Q605-templates were diluted to 1-10 pM in 1% BSA/PBS, pipetted into flow cell lanes, and allowed to bind to the streptavidin-modified surface for 30-60 min, followed by five PBST washes. Flow cell lanes were then equilibrated with Extension Buffer (50 mM Tris pH 8.0, 50 mM NaCl, 10 mM MgCl₂), followed by outfitting the flow cell with inlet and outlet tubing for subsequent fluid deliver and mounting on the TIRF microscope system.

Biotinylated-Q605-templates were focused in the TIRF plane and polymerization reaction components (5 μM [AF647]-γ-deoxyguanosine tetraphosphate and 0.02 U/μL exo⁻ Klenow fragment in Extension Buffer) were injected into the flow cell lane through the inlet tubing while live video was collected at ˜30 frames/sec for 5-10 min.

TIRF microscope system for single-molecule fluorescence detection. The TIRF system was employed using Olympus IX71 inverted frame series. 405 nm laser, 60× PLAN APO Objective, 1.45 N.A., dichroic mirror in the turret is SEMROCK FF510 which reflects wavelengths below 510 nm. Olympus USIP image splitter was used with emission filters from SEMROCK. The Camera is a Hamamatsu C9100-13 EMCCD camera. This measurement was performed at 30 frames/second, with gain of 255.

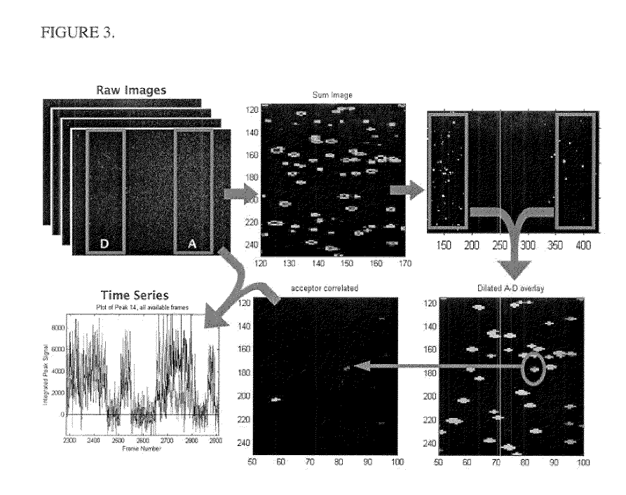

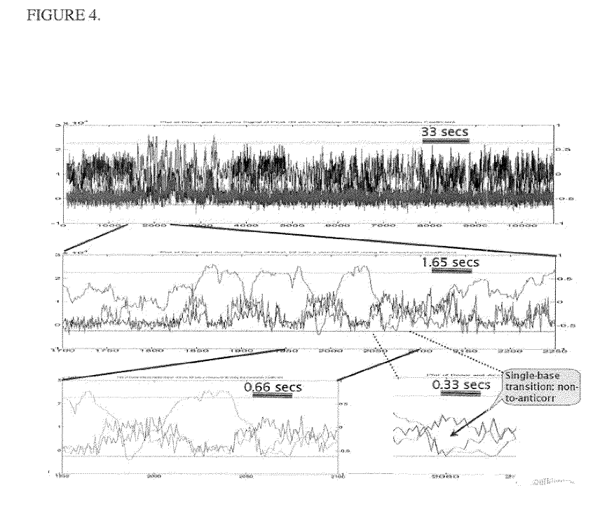

Image analysis. Data analysis was performed as followed (see FIG. 3). The 5-dimensional data set of Fluorescence vs. x-axis of microscope slide, y-axis of microscope slide, 605 nm±15 nm, 670 nm±30 nm, and time was analyzed by: (1) first locating the x-coordinate and y-coordinate locations of the quantum dot nanocrystal donor signal (at 605 nm). The corresponding acceptor-dye fluorescence x, y, position was thereby set as 256 pixels translated along the y-axis of the 5-dimensional data set (relationship established by the dual-view 2-color image splitter utilized by this instrument). Time-series data was thereby extracted from the 5-dimensional data set and examined for correlated signal changes in the donor and acceptor color channels (see FIG. 3). The time-dependent correlation between the donor and acceptor signal can be directly calculated as the mathematical inner product of the donor-acceptor time series. This normalized inner product (value ranged from −1 (negative correlation) to +1 (positive correlation) can be plotted superimposed on the original time-series data. Standard confidence interval calculations can be utilized to set the confidence limits of the positive and negative correlation (confidence limits of 99.99% were set for these data sets). For a base insertion event, it is predicted that one would observe a series of changes in correlation in the following sequence: non-correlated signals followed by anticorrelation (initial dye-dNTP binding event, donor signal decreases and acceptor signal increases) followed by positive correlation (while dye-dNTP is bound to DNA polymerase) followed by anticorrelation (donor signal increases and acceptor signal decreases back to baseline) as the dye-phosphate diffuses of the polymerase. Such patterns are indeed found in these time series (FIG. 4).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method for genotyping or sequencing a single target nucleic acid molecule, said method comprising:
a) immobilizing onto a solid support a target nucleic acid molecule to form a solid support comprising more than one site or location each bearing only one individual molecule of target nucleic acid sequence;
b) contacting the solid support with a polymerase, a primer operably linked to at least one semiconductor nanocrystal, and at least one fluorescent labeled nucleotide polyphosphate;
c) optically detecting a time sequence of incorporation of the fluorescently labeled nucleotide polyphosphates into the growing nucleotide strand at an active site complementary to the target nucleic acid molecule, by detecting fluorescence resonance energy transfer (FRET) signals between the semiconductor nanocrystal and the fluorescent labeled nucleotide polyphosphate,
wherein the identity of each fluorescent labeled nucleotide is determined by its fluorescent label,
wherein the fluorescent label is then cleaved from the nucleotide upon incorporation into the growing strand; and
d) genotyping or sequencing said single target nucleic acid molecule by converting the sequence of the FRET signals detected during the polymerization reaction into a nucleic acid sequence.

2. The method of claim 1, wherein the target nucleic acid molecule is DNA, and the polymerase is a DNA or RNA polymerase.

3. The method of claim 1, wherein the target nucleic acid molecule is RNA, and the polymerase is reverse transcriptase.

4. The method of claim 1, wherein the polymerase is a Klenow fragment of DNA polymerase I, E. coli DNA polymerase I, T7 DNA polymerase, T4 DNA polymerase, Thermus aquaticus DNA polymerase, or Thermococcus litoralis, DNA polymerase.

5. The method of claim 1, wherein the semiconductor nanocrystal acts as a donor fluorophore and the fluorescent label on the nucleotide polyphosphate acts as the acceptor fluorophore.

6. The method of claim 1, wherein the fluorescent label is selected from the group consisting of fluorescein, cyanine, rhodamine, coumarin, acridine, Texas Red dye, BODIPY, ALEXA, and a derivative or modification of any of the foregoing.

7. The method of claim 1, wherein the fluorescent label is attached to the γ-phosphate of the nucleotide polyphosphate.

8. The method of claim 1, wherein the detection occurs in real-time or near real-time.

9. The method of claim 1, further comprising sequencing a second nucleic acid according to the method of claim 1 in parallel with sequencing the first nucleic acid.

10. The method of claim 1, wherein the solid support is glass or plastic.

11. The method of claim 4, wherein the primer is extended by a plurality of nucleotides.

12. The method of claim 5, wherein the primer is extended by less than 50 nucleotides.

13. The method of claim 1, wherein the primer comprises at least 10 nucleotides.

14. The method of claim 1, wherein the primer comprises at least 20 nucleotides.

15. The method of claim 1, wherein the fluorescent labeled nucleotide polyphosphate has three or more phosphates.

16. The method of claim 1, wherein the fluorescent labeled nucleotide polyphosphate has four or more phosphates.


SEQUENCE LISTING (1 sequence)

SEQ ID NO: 1 - DNA, 31 bp, Artificial Sequence
  Oligonucleotide
tttttgagggtgacaggtttttcctgtcacc